(19) United States
(12) Patent Application Publication
(10) Pub. No.: US 2017/0143594 A1
(43) Pub. Date: May 25, 2017

(54) ORTHODONTIC CEMENT COMPOSITIONS AND METHODS OF USE THEREOF

(71) Applicant: DENTSPLY SIRONA Inc., York, PA (US)

(72) Inventors: Hui LU, Magnolia, DE (US); Xiaoming JIN, Middletown, DE (US)

(21) Appl. No.: 15/356,726

(22) Filed: Nov. 21, 2016

Related U.S. Application Data
(60) Provisional application No. 62/258,123, filed on Nov. 20, 2015.

(51) Int. Cl. A61K 6/083; A61C 7/16; A61C 13/15; A61K 6/00

(52) U.S. Cl. CPC A61K 6/0835; A61K 6/0091; A61K 6/0052; A61K 6/0067; A61C 7/16; A61C 19/003

(57) ABSTRACT

Disclosed herein is an orthodontic cement having an effective polymerizable antibacterial resin that is capable of balanced antibacterial effectiveness, prevention of white spot lesions during orthodontic treatment, and excellent adhesion properties. Disclosed is a method and orthodontic cement composition comprising polymerizable antibacterial/antimicrobial monomers, and a high performance orthodontic cement formulated from such novel bioactive resins.

### BACKGROUND

Disclosed herein are compositions related to light-curable orthodontic cements that can adhere orthodontic appliances to human tooth structures, as well as providing adequate antibacterial functions to prevent or mediate demineralization and occurrence of white spot lesions (WSL).

The developments of incipient carious lesions around orthodontic brackets are one of the most common undesirable outcomes during the orthodontic treatment using fixed appliances. These white spot lesions may also have lasting negative impacts on the overall aesthetics, even after certain post-orthodontic intervention. Depending on the diagnostic techniques used, the prevalence of WSL can vary widely according to the published literatures. It has been reported that between 36% to 89% of patients using fixed orthodontic appliances exhibited various levels of carious lesions during the orthodontic treatment.

Maxillary teeth are most commonly affected with the order of incidence being lateral incisors, canines, premolars, and central incisors. Due to larger, more retentive surface area introduced by brackets and other fixed orthodontic appliances and their irregular shapes, it became quite challenging to maintain good oral hygiene, especially for “non-compliant” or “poor-compliant” patient groups. Therefore, higher occurrences for plaque/biofilm adherence during the intra-treatment tend to occur and eventually lead to caries lesions, which could become more severe for patients having higher caries risk even before the orthodontic treatment.

Additionally, it has been reported that fixed orthodontic appliances can affect the self-cleaning capabilities of teeth, owing to the interactions of saliva, tongue, and teeth surfaces. The fixed orthodontic appliances can even alter the oral microflora and increase the levels of acidogenic plaque bacteria, i.e. Streptococci mutans (S. mutans) and lactobacilli in saliva and dental biofilm during active wear of the appliance⁶⁻¹¹.

Due to the extensive prevalence of white spot lesion occurred during the orthodontic treatments, various strategies have been proposed to prevent or mediate demineralization and occurrence of WSL formation. Depending on patient compliance, the approaches range from mechanical removal of plaque/biofilm, to use of fluoride in various forms (topical varnish, mouth rinse, tooth paste, etc), to the use of antimicrobial agents such as Xylitol.

The findings consolidated by Bergstrand and Twetman¹² concluded that the use of topical fluorides in addition to fluoride toothpaste as the best evidence-based way to prevent WSL. The mean prevented fraction based on 6 clinical trials was 42.5% with a range from 4% to 73%. The findings provided the one of the strongest support for regular professional applications of fluoride varnish around the bracket base during the course of orthodontic treatment. For the treatment of post-orthodontic WSL, home-care applications of a remineralizing cream, based on casein phosphopeptide-stabilized amorphous calcium phosphate, as adjunct to fluoride toothpaste could be beneficial but the findings were equivocal. For emerging technologies such as sugar alcohols and probiotics, still only studies with surrogate endpoints are available. Thus, further well-designed studies with standardized regimes and endpoints are needed before guidelines on the non-fluoride technologies can be recommended. In general, fluoride has shown some benefit as a protective measure against demineralization; however, they could be insufficient for orthodontic patients with less than ideal oral hygiene.

Another class of material that have attract significant research for anti-WSL application is amorphous calcium phosphate (ACP) and calcium sodium phosphosilicate. According to Dr. Heymann and Dr. Grauer, ACP is thought to have the potential to both prevent and mediate enamel demineralization in patients with high caries risk. Dentifrices containing calcium sodium phosphosilicate bioactive glass (NovaMin) have been proposed to aid in prevention of white spot lesions and gingival inflammation. Hoffman et al. ¹⁴ conducted prospective, double-blind, randomized controlled trial. The study included control group consisted of 24 patients who received over-the-counter fluoride toothpaste (Crest®), while the study group consisted of 24 patients who were given the test dentifrice (ReNew™) containing 5% NovaMin and fluoride. Patients were followed up for 6 months on a monthly basis. However, they reported that there were no significant differences between the groups in regard to changes in white spot lesions, plaque, or gingival health (P>0.05). There was a trend toward improvement in white spot lesions found in subjects using Crest® at the 3-month time point. This was not sustained throughout the study. The authors concluded that toothpaste containing NovaMin does not differ significantly compared to traditional fluoride toothpaste for improving white spot lesions and gingivitis in orthodontic patients.

There are also products such as MI Paste (GC) contains casein phosphopeptide—amorphous calcium phosphate (CPP-ACP), a milk-derived protein that helps to promote high rates of enamel remineralization. MI Paste Plus is the same product, but also contains 900 ppm of fluoride. A recent randomized controlled trial demonstrated that orthodontic patients who applied MI Paste Plus nightly via a fluoride delivery tray for 3 to 5 minutes following brushing showed fewer and less severe WSL than controls¹³. It has been suggested that ACP may aid in the remineralization of WSL after the completion of orthodontic treatment, although there is some evidence that shows no significant advantage for use of ACP supplementary to normal oral hygiene. That is, there was no significant difference in the reduction of WSL size between patients who used MI Paste and those who used regular oral hygiene including 1,000-ppm toothpaste.

It has been well recognized that dental caries or decays are closely associated with the cariogenic bacterial contained in dental biofilm, more specifically, as a result of demineralization of tooth structure due to the acid produced by bacteria such as S. mutans in the presence of fermentable carbohydrates. S. mutans are one of few specialized organisms equipped with receptors that can improve adhesion to the surface of teeth. Sucrose is used by S. mutans to produce a sticky, extracellular, dextran-based polysaccharide that allows them to cohere, colonize, and form dental plaque. The combination of plaque and acid leads to dental decay.

Dental biofilm is a highly complex, heterogeneous, and dynamic structure. Up to 500 different bacterial species have been identified in human oral biofilm. For oral and systemic health, the dental biofilm needs to be regularly and meticulously removed. Removal and reduction of biofilm can be achieved by mechanical means, chemical means, or combination. There have been increasing efforts to inhibit the development of dental biofilm. It is known prior to the development of dental biofilm, the salivary or acquired pellicle forms. This occurs through the adsorption of protein from saliva onto the clean tooth surface. Acquired pellicle formation provides oral bacterial with biding sites, resulting in bacterial adhesion, the first step in the formation of dental biofilm. Therefore, surface modification should inhibit the development of the acquired pellicle and dental biofilm.

In restorative dentistry, extensive attempts have been made to create dental compositions with antibacterial/antimicrobial effects, by incorporation of a variety of antibacterial/antimicrobial agents, such as chlorhexidine, silver ions, zinc ions, and fluoride, etc. Although such low molecular compounds demonstrated certain immediate effectiveness, there are controversial related to their long-term effectiveness, esthetics, potential toxicity, and impact to the mechanical strength of the formulated dental composition due to the leachability. On the other hand, solid antibacterial/antimicrobial agents such as silver nanoparticles and polymeric quantum ammonium salt (QAS) nanoparticles were also developed to address those issues associated with the low molecular weight of antibacterial/antimicrobial agents. There are also issues such as color, optical opacity, and mechanical strength. Recently polymerizable antibacterial/antimicrobial resins were developed but their sub-optimal effectiveness require relatively high loading level, and most of them demonstrated negative impact on mechanical property in the formulated dental compositions, with the increased concentration.

U.S. Publication No. 2010/0256242 disclosed a polymerizable biomedical composition that includes a quaternary ammonium group bonded at its quaternary sites.

U.S. Pat. No. 5,494,987 disclosed antimicrobial polymerizable compositions having an ethylenically unsaturated monomer with antimicrobial activity for dental application composed of quaternary ammonium dodecylpyridinium (MDPB).

U.S. Pat. No. 8,236,337 disclosed anti-microbial orthodontic apparatus and anti-microbial orthodontic compositions comprising an effective amount of a selenium compound.

U.S. Pat. Nos. 6,710,181 and 7,094,845 disclosed an imidazole-based silane and monocarboxylic acid salt for improving adhesion between resins and metal or glass.

U.S. Pat. No. 7,553,881 disclosed dental compositions based on polymerizable macromers based on quaternary ammonium salts for antimicrobial effect.

U.S. Pat. No. 8,747,831 disclosed dental composition and method of making a polymerizable antibacterial/ antimicrobial resin and using such a bioactive resin in formulated dental compositions.

### SUMMARY

In summary, there is strong need to develop orthodontic cement having highly effective polymerizable antibacterial resin that is capable to offer a balanced antibacterial effectiveness, prevention of WSL during the orthodontic treatment, and excellent adhesion properties. In this invention, a method and orthodontic cement composition comprising polymerizable antibacterial/antimicrobial monomers is disclosed and high performance orthodontic cement are formulated from such novel bioactive resins. The polymerizable antibacterial/antimicrobial monomers The polymerizable antibacterial/antimicrobial monomers contain at least one or multiple imidazolium groups and at least one or multiple radically polymerizable groups as shown in the following formula:

(M-X1)ₘ-A-(CB-I-X2)ₙ

M: free radical polymerizable moiety such as acrylamide, methacrylamide, vinyl, acrylate, methacrylate, etc.

X1, X2: equal or different moieties, such as alkyl, aromatic, amide, ether, ester, direct etc,

A: moieties such as aromatic or alkyl;

C: counter ion moieties such as bromine, iodine, chlorine, halogen atom, etc

B: moieties containing alkyl group having 0-15 carbon atoms;

I: imidazole moiety or substituted imidazole moiety like imidazole, methyl-imidazole, where m and n are integers of at least 1.

### DETAILED DESCRIPTION

Described herein are orthodontic cement compositions designed to not only function as a conventional cementation agent to fix orthodontic appliances (such as bracket) onto tooth surface, but also provide antimicrobial functions to prevent/mitigate the occurrence of bacterial induced demineralization and white spot lesions. The composition in the described invention also has good physical properties and it can be cured by both a traditional quartz-tungsten-halogen (QTH) dental lamp and a light emitting diode (LED) dental lamp.

The copolymerizable multi-functional (meth)acrylate monomer may be a free radically polymerizable compound, such as mono-, di- or multi-methacrylates and acrylates such as methyl methacrylate, isopropyl methacrylate, ethyl acrylate, triethyleneglycol dimethacrylate, n-hexyl acrylate, stearyl acrylate, allyl acrylate, glycerol diacrylate, glycerol triacrylate, ethyleneglycol diacrylate, diethyleneglycol diacrylate, tetraethylene glycol di(meth)acrylate, 1,3-propanediol diacrylate, 3-(acryloyloxy)-2-hydroxypropyl methacrylate, 1,3-propanediol dimethacrylate, trimethylolpropanetri(meth)acrylate, 1,2,4-butanetriol trimethacrylate, 1,4-cyclohexanediol diacrylate, 1,6-hexanediol di(meth)acrylate, pentaerythritol triacrylate, pentaerythritol tetraacrylate, pentaerythritol tetramethacrylate, sorbitol hexacrylate, 2,2-bis [4-(2-hydroxy -3 -acry loyloxypropoxy) phenyl]propane; 2,2-bis [4-(2-hydroxy-3-methacryloy loxypropoxy)phenyl] propane (Bis-GMA); 2,2-bis[4-(acryloyloxy-ethoxy)phenyl] propane; 2,2-bis[4-(methacryloyloxy-ethoxy)phendyl] propane (or ethoxylated bisphenol A-dimethacrylate) (EBPADMA), polycarbonate dimethacrylate (PCDMA), 2,7,7,9,15-pentamethy-4, 13 dioxo-3,14 dioxa-5,12-diaza hexadecane-1, diyldimethacrylate, urethane di(meth)acrylate (UDMA), bis-acrylates and bis-methacrylates of polymethylene glycols. The copolymerizable multi-functional (meth)acrylate monomer may be present in the composition in an amount of from about 50 weight percent to about 95 weight percent of the resin matrix, such as from about 60 weight percent to about 90 weight percent or from about 65 weight percent to about 90 weight percent of the resin matrix.

It was found a variety of polymethacrylated resins containing at least one polyimidazole moiety could be readily prepared by appropriate hybrid acrylate-methacrylate resins or polyacrylate resins with proper control of the conversion of the imidazole addition. This is an effective method to incorporate an imidazole moiety into a polymerizable resin as novel, acid-free functional resins. Furthermore, such polymerizable imidazole-containing resins may be further chemically modified by reaction with a variety of halogenated alkyls to form polymerizable resins with ionic moiety of imidazolium, which should be new class of polymerizable ionic liquid resins. Since organic compounds incorporating such imidazolium moieties are commonly utilized as antibacterial/antimicrobial agents, it was expected the polymerizable imidazolium-based resins could also exhibit highly effective bactericidal functions and could further prevent or mediate the occurrence of white spot lesion triggered by cariogenic bacterial such as S mutans.

A variety of polymethacrylate resins with polyimidazoles are able to be prepared by coupling with different mono, di, tri, or polyols or polyamines. Further, in order to streamline the process of making such imidazole-based polymerizable resins for use in making imidazolium-based polymerizable resins, a facile process based on imidazole and acrylated resins were investigated as illustrated in Scheme 1. Thus a variety of imidazole-containing polymerizable monomers are able to be prepared accordingly as examples shown in Scheme 2 and Scheme 3.

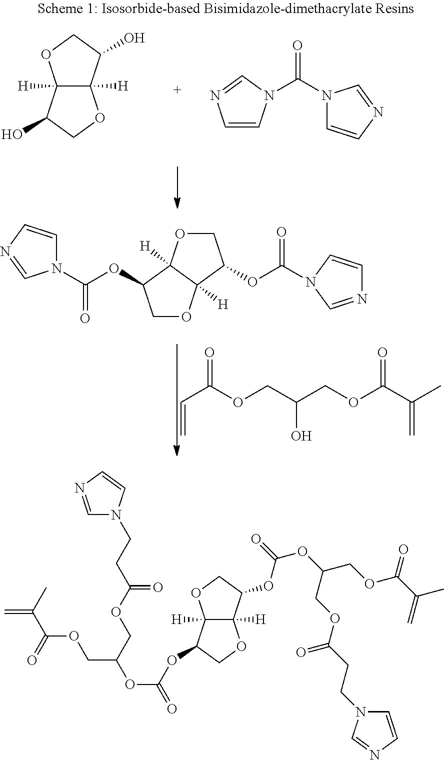

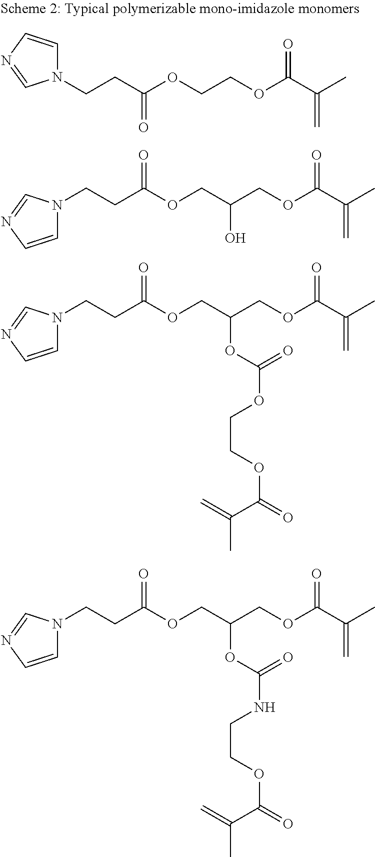

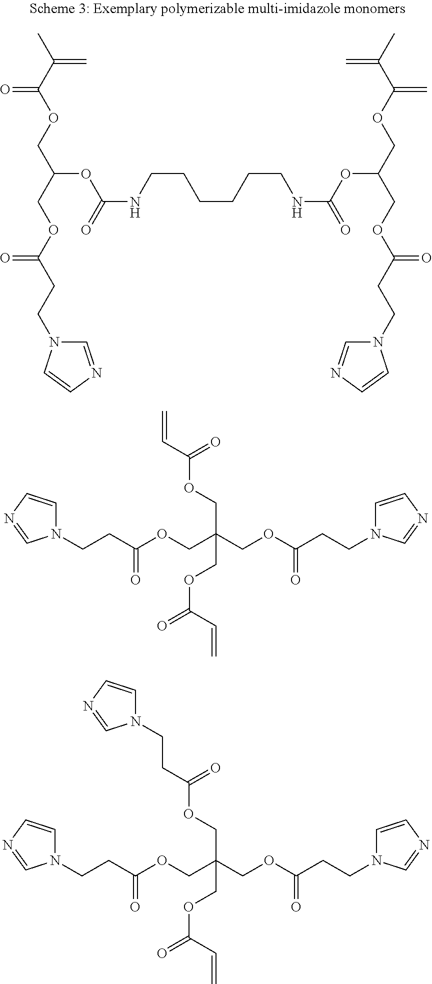 [SMILES: C=C(C)C(=C)OCC(COC(=O)CCn1ccnc1)OC(=O)NCCCCCCNC(=O)OC(COC(=O)CCn1ccnc1)COC(=O)C(=C)C ; C=CC(=O)OCC(COC(=O)C=C)(COC(=O)CCn1ccnc1)COC(=O)CCn1ccnc1 ; C=CC(=O)OCC(COC(=O)CCn1ccnc1)(COC(=O)CCn1ccnc1)COC(=O)CCn1ccnc1]

The preferred imidazolium-containing polymerizable monomer contains at least one polymerizable group such as methacrylate or acrylate and at least one imidazolium moiety bearing linear long alkyl chain of C8-C14. The most preferred resin contains two methacrylate group and at least one imidazolium moiety bearing C12 linear alkyl chain.

Dental composition disclosed herein may be composed of (1) the functional polymerizable resins contains imidazole group or imidazolium groups described herein in amount of from about 0.5 weight percent to about 99 weight percent of the dental composition, (2) conventional polymerizable resin in amounts of from about 10 weight percent to about 99 weight percent of the dental composition, (3) initiators and other additives in amounts of from about 0.001 weight percent to about 5.0 weight percent of the dental composition, (4) a plurality of filler particles having a size of from about 10 nm to about 100 micron of the dental composition, and (5) an optional inert solvent in amounts not to exceed 1 weight percent of the dental composition.

HEMA and HPMA are typical monomethacrylate resins; BisGMA, TEGDMA, UDMA are typical conventional dimethacrylate resins, which are polymerizable/curable by heat, light and redox initiation processes. CQ and LTPO are typical photoinitiators. Tertiary aromatic amines, such as EDAB, may be included as an accelerator for CQ-based photoinitiator. Other additives such as inhibitors, UV stabilizers or flourencent agents may also be used. In addition, a variety of particles, polymeric, inorganic, organic particles may be incorporated to reinforce the mechanical properties, rheological properties and sometime biological functionalities.

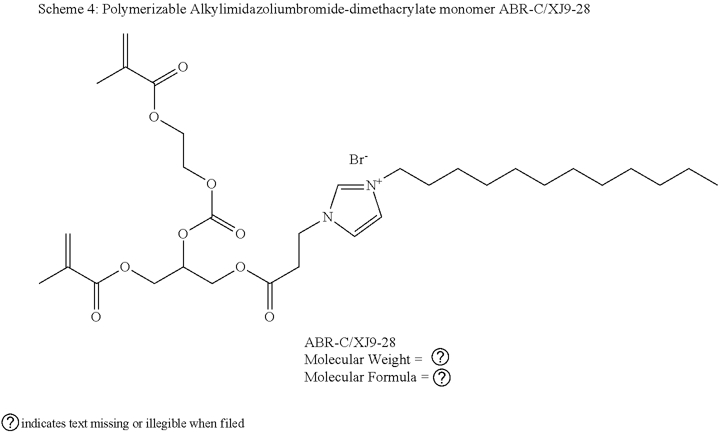 [SMILES: C=C(C)C(=O)OCCOC(=O)OC(COC(=O)CCn1cc[n+](CCCCCCCCCCCC)c1)COC(=O)C(=C)C ; [Br-]]

The antibacterial orthodontic cement composition disclosed herein further comprises one or more types of filler particles that are suitable for use in dental compositions. Filler particles are critical components to the composition described herein. Fillers that are suitable for use in the composition described herein provide the composite with desired physical and curing properties, such as increased strength, modulus, hardness, reduced thermal expansion and polymerization shrinkage, and also provide a stable shelf life such that no adverse reaction occurs between the filler particles with any of the resin matrix's organic compounds in composition during storage or transportation, and before the intended shelf-life is reached.

Examples of suitable filler particles include, but are not limited to, strontium silicate, strontium borosilicate, barium silicate, barium borosilicate, barium fluoroalumino borosilicate glass, barium alumino borosilicate, calcium silicate, calcium alumino sodium fluoro phosphor-silicate lanthanum silicate, alumino silicate, and the combination comprising at least one of the foregoing fillers. The filler particles can further comprise silicon nitrides, titanium dioxide, fumed silica, colloidal silica, quartz, kaolin ceramics, calcium hydroxy apatite, zirconia, and mixtures thereof. Examples of fumed silica include OX-50 from DeGussa AG (having an average particle size of 40 nm), Aerosil R-972 from DeGussa AG (having an average particle size of 16 nm), Aerosil 9200 from DeGussa AG (having an average particle size of 20 nm), other Aerosil fumed silica might include Aerosil 90, Aerosil 150, Aerosil 200, Aerosil 300, Aerosil 380, Aerosil R711, Aerosil R7200, and Aerosil R8200, and Cab-O-Sil M5, Cab-O-Sil TS-720, Cab-O-Sil TS-610 from Cabot Corp.

The filler particles used in the composition disclosed herein may be surface treated before they are blended with organic compounds. The surface treatment using silane coupling agents or other compounds are beneficial as they enable the filler particles to be more uniformly dispersed in the organic resin matrix, and also improve physical and mechanical properties. Suitable silane coupling agents include 3-methacryloxypropyltrimethoxysilane, methacryloxyoctyltrimethoxysilane, styrylethyltrimethoxsilane, 3-mercaptopropyltrimethoxysilen, and mixtures thereof.

Fillers may be present in amounts of from about 40 weight percent to about 85 weight percent of the antibacterial orthodontic cement composition, such as from about 45 weight percent to about 85 weight percent or from about 60 weight percent to about 80 weight percent of the antibacterial orthodontic cement composition.

The filler particles can have a particle size of from about 0.002 microns to about 25 microns. In one embodiment, the filler can comprise a mixture of a micron-sized radiopaque filler such as barium alumino fluoro borosilicate glass (BAFG, having an average particle size of about 1 micron) with nanofiller particles, such as fumed silica such as OX-50 from DeGussa AG (having an average particle size of about 40 nm). The concentration of micron-size glass particles can range from about 50 weight percent to about 75 weight percent of the antibacterial orthodontic cement composition, and the nano-size filler particles can range from about 1 weight percent to about 20 weight percent of the antibacterial orthodontic cement composition.

The antibacterial orthodontic cement composition described herein further contains a polymerization initiator system. The initiator is not particularly limited and may be a photoinitiator. The present composition may employ a dual-photoinitiator system having camphorquinone (CQ) and diphenyl (2,4,6-trimethylbenzoyl) phosphine oxide (L-TPO), which proves to be an effective combination in an effective concentration to be compatible with an amine polymerization accelerator, as described below.

The polymerization photoinitiators (the combination of CQ and L-TPO) are present in an amount of from about 0.05 weight percent to about 1.00 weight percent, such as from about 0.08 weight percent to about 0.50 weight percent or from about 0.10 weight percent to about 0.25 weight percent of the antibacterial orthodontic cement composition. Using such a small amount of a polymerization photoinitiators decreases the potential discoloration of the composition. By contrast, compositions containing a high concentration a photoinitiator are more likely to be discolored.

Other diketone type photoinitiator such as 1-phenyl-1,2 propanedione (PPD), and phosphine oxide type photoinitiator such as Ciba-Geigy's bis(2,4,6-trimethylbenzoyl)-phenylphospohine oxide (Irgacure 819), BASF's ethyl 2,4,6-trimethylbenzylphenyl phosphinate (Lucirin LR8893X), may also be used.

The polymerization initiator system of the antibacterial orthodontic cement composition described herein may further include a polymerization accelerator, which may be a tertiary amine. One example of a suitable tertiary amine is ethyl 4-(dimethylamino)benzoate (EDAB). Other tertiary amines that may be used include 2-(ethylhexyl)-4-(N,N-dimethylamino)benzoate, dimethyl aminobenzoic acid ester, triethanol amine, N,N,3,5,N,3,5-tetramethyl aniline, 4-(dimethyl amino)-phenethyl alcohol, dimethyl aminobenzoic acid ester, 4-(N,N-dimethylamino)benzoic acid, sodium benzenesulfinate, and the like.

The polymerization accelerator may be present in an amount of from about 0.03 weight percent to about 0.18 weight percent of the antibacterial orthodontic cement composition, such as from about 0.04 weight percent to about 0.15 weight percent or from about 0.05 weight percent to about 0.12 weight percent of the antibacterial orthodontic cement composition. The compositions disclosed herein are capable of being activated by a curing light having a wavelength of from about 380 nm to about 500 nm.

The antibacterial orthodontic cement composition described herein may further include additives in order to provide specifically desired features. These additives include ultra-violet stabilizers, fluorescent agents, opalescent agents, pigments, viscosity modifiers, fluoride-releasing agents, polymerization inhibitors, and the like. Typical polymerization inhibitors for a free radical system may include hydroquinine monomethyl ether (MEHQ), butylated hydroxytoluene (BHT), tertiary butyl hydro quinine (TBHQ), hydroquinone, phenol, butyl hydroxyanaline, and the like. The inhibitors act as free radical scavengers to trap free radicals in the composition and to extend the shelf life stability of the composition. The polymerization inhibitors, if present, may be present in amounts of from about 0.001 weight percent to about 1.5 weight percent of the antibacterial orthodontic cement composition, such as from about 0.005 weight percent to about 1.1 weight percent or from about 0.01 weight percent to about 0.08 weight percent of antibacterial orthodontic cement composition. The composition may include one or more polymerization inhibitors.

The antibacterial orthodontic cement composition disclosed herein may be made by any known and conventional method. In embodiments, the composition is made by mixing the components together at a temperature of from about 20° C. to about 60° C., such as from about 23° C. to about 50° C. The monomers, photoinitiators, accelerators, and other additives can be blended first to form a paste of a uniform mixed resin blend. The paste can be prepared by mixing the components for a total of about 30 seconds to about 5 minutes, such as from about 1 minute to about 3 minutes or about 1.5 minutes, on a speedmixer, such as a Flack-Tec at room temperature (from about 23° C. to about 27° C.), followed by further mixing in a Ross Mini Mixer that is equipped with temperature and vacuum control, for a time of from about 20 minutes to an hour, such as from about 30 minutes to 50 minutes or about 40 minutes, under from about 20 to about 27 inches Hg vacuum at room temperature (from about 23° C. to about 27° C.) or further mixing in the Ross Mini Mixer takes place for a time of from about 10 minutes to about 30 minutes, such as from about 15 minutes to about 25 minutes or about 20 minutes, under from about 20 to about 27 inches Hg vacuum at an elevated temperature of from about 40° C. to about 60° C., such as from about 45° C. to about 55° C. or about 50° C. In alternative embodiments, the paste may be mixed in a Ross Mini Mixer for a time of from about 40 minutes to an hour, under from about 20 to about 27 inches Hg vacuum at an elevated temperature of from about 40° C. to about 60° C., such as from about 45° C. to about 55° C. or about 50° C., without initially using a speedmixer, as described. In yet further embodiments, the paste may be mixed on Resodyn Acoustic Mixer for a time of from about 30 minutes to about 60 minutes, such as from about 35 minutes to about 55 minutes or about 45 minutes under from about 20 to about 27 inches Hg vacuum at a temperature of from about 18° C. to about 30° C., such as 20° C. to about 27° C. or 23° C.

### DETAILED DESCRIPTION

### Test Methods

ISO-22196 Antimicrobial Test: This test was conducted at Antimicrobial Test Laboratories (Round Rock, Tex.), an independent and GLP complied testing institution. ISO method 22196 is a quantitative test designed to assess the performance of materials' antimicrobial capabilities on hard, non-porous surfaces. The method can be conducted using contact times ranging from ten minutes up to 24 hours. For a ISO 22196 test, non-antimicrobial control surfaces are used as the baseline for calculations of microbial reduction. The test microorganism selected for this test is Staphylococcus aureus 6538 (S. aureus 6538). This bacterium is a Gram-positive, spherical-shaped, facultative anaerobe. Staphylococcus species are known to demonstrate resistance to antibiotics such as methicillin. S. aureus pathogenicity can range from commensal skin colonization to more severe diseases such as pneumonia and toxic shock syndrome (TSS). S. aureus is commonly used in standard test methods as a model for gram positive bacteria.

Summary of the ISO-22196 Antimicrobial Procedure:

The test microorganism is prepared, usually by growth in a liquid culture medium.

The suspension of test microorganism is standardized by dilution in a nutritive broth (this affords microorganisms the opportunity to proliferate during the test).

Control and test surfaces are inoculated with microorganisms, and then the microbial inoculum is covered with a thin, sterile film. Covering the inoculum spreads it, prevents it from evaporating, and ensures close contact with the antimicrobial surface.

Microbial concentrations are determined at “time zero” by elution followed by dilution and plating to agar.

A control is run to verify that the neutralization/elution method effectively neutralizes the antimicrobial agent in the antimicrobial surface being tested.

Inoculated, covered control and antimicrobial test surfaces are allowed to incubate undisturbed in a humid environment for 24 hours, usually at body temperature.

After incubation, microbial concentrations are determined. Reduction of microorganisms relative to the control surface is calculated.

Notched-Edge Shear Bond Strength: Freshly extracted, caries-free and un-restored human molars were used. Teeth were sectioned longitudinally through the mesial, occlusal, and distal surfaces using a water-cooled diamond grit cutting disc. The sectioned molars were then mounted in a cylindrical block using cold-cure acrylics, with the buccal surface exposed. The exposed surface was then coarse ground on a model trimmer until a flat dentin or enamel surface was exposed. Prior to the bonding of specimen, tooth was wet-ground on grinding wheel under running water use 120-grit SiC sanding paper, followed by 320-grit SiC sanding paper, until the surface is even and smooth when visually inspected. The Notched-Edge bonding jig contains a cylindrical plastic mold resulting in samples with a defined bonding area (diameter 2.38 mm). The herein described antibacterial orthodontic cement restorative composite is then carefully placed into the center of the mold, without any bonding agent or primer being applied to the substrate first. After light curing 550 mW/cm² for 20 seconds, the specimen was then carefully removed from mold. Specimens were stored in 37° C. DI-water for 24 hour before SBS testing. SBS test was performed on Instron Universal Tester 4400R at a crosshead speed of 1 mm/min. A minimum of seven specimens were tested for each set of sample.

Flexural Strength: Specimens for 3-point bending flexural test were prepared according to ISO 4049. Sample were filled into 25 mm×2 mm×2 mm stainless steel mold, then covered with Mylar film and cured using Spectrum 800 (DENTSPLY Caulk) halogen lamp at intensity of 550 mW/cm² for 4X20 seconds uniformly across the entire length of the specimen. The set specimens were stored in deionized water at 37° C. for 24 hours prior to the test. Flexural test was conducted using an Instron Universal Tester Model 4400R with crosshead speed 0.75 mm/min under compressive loading mode. A minimum of six specimens were tested for each set of sample.

Compressive Strength: Samples were filled into Ø4×7 mm Teflon molds and sandwiched between two Mylar cover films, then cured using Spectrum 800 lamp at intensity of 550 mW/cm² on both ends. The set specimens were stored in deionized water at 37° C. for 24 hours prior being polished to 6 mm long×4 mm in diameter using 600 grit sand paper. Compression test was conducted using an Instron Universal Tester Model 4400R with crosshead speed 5 mm/min. Six specimens were tested for each set of sample.

### EXAMPLES

The following abbreviations may be used herein:

UDMA: di(methacryloxyethyl)trimethyl-1,6-hexaethylenediurethane

BisGMA: 2,2-bis(4-(3-methacryloyloxy-2-hydroxypropoxy)-phenyl)propane

PENTA: Dipentaerythritol pentaacrylate phosphoric acid ester

TMPTMA: Trimethylolpropane Trimethacrylate

TCDC: 4,8-bis(hydroxymethyl)-tricyclo[5,2,1,0]

TEGDMA: triethylene glycol dimethacrylate

HPMA: 2-hydroxypropyl methacrylate

CDI: 1,1-carbonyl-diimidazole

HEMA: 2-hydroxyethyl methacrylate

SR295: pentaerythritol tetraacrylate

AMAHP: 3-(acryloyloxy)-2-hydroxypropyl methacrylate

EGAMA: ethyleneglycol acrylate methacrylate

CQ; camphorquinone

L-TPO: lucirin TPO/2,4,6-trimethylbenzoyldiphenylphosphine oxide

EDAB: 4-Ethyl dimethylaminobenzonate

BHT: butylhydroxytoluene

Silanated BFBG-1: barium fluoroalumino borosilicate glass surface treated by γ-methacryloxypropyltrimethoxysilane

Silanated BFBG-2: barium fluoroalumino borosilicate glass surface treated by γ-methacryloxypropyltrimethoxysilane

Silanated SAFG: Silanated Strontium-AluminoSodium-Fluoro-Phosphorsilicate glass surface treated by γ-methacryloxypropyltrimethoxysilane

A variety of antibacterial orthodontic resin (without inorganic fillers) and antibacterial orthodontic cement (contains inorganic fillers) composition were prepared, and their properties have been evaluated.

### Comparable Example 1

Conventional light curable orthodontic resin (HLU14-114-SO), without antibacterial monomer, was formulated, tested, and used as control resin.

### Example 1

light curable orthodontic resin (HLU14-196R1) that contains 4 wt % polymerizable antibacterial monomer (ABR-C/XJ9-28, Scheme 4) in resin was formulated and a homogeneous resin mixture was obtained;

### Example 2

light curable orthodontic resin (HLU14-182R) that contains 8 wt % polymerizable antibacterial monomer (ABR-C/XJ9-28) in resin was formulated and a homogeneous resin mixture was obtained;

### Example 3

light curable orthodontic resin (HLU14-196R2) that contains 12 wt % polymerizable antibacterial monomer (ABR-C/XJ9-28) in resin was formulated and a homogeneous resin mixture was obtained;

### Example 4

light curable orthodontic resin (HLU14-183R) that contains 16 wt % polymerizable antibacterial monomer (ABR-C/XJ9-28) in resin was formulated and a homogeneous resin mixture was obtained;

As shown in Table 1 and FIG. 1-2, up to 12 wt % loading level, there are no significant decreases of flexural strength or modulus with the incorporation of the imidazole-based, polymerizable antibacterial monomer (ABR-C/XJ9-28) observed, as compared to control (Comparable Example 1). The flexural strength of resin mixture (Example 4) showed lower flexural strength but flexural modulus still retains 87% value as compared to control.

TABLE 1
Flexural Strength and Modulus of orthodontic resins that contain various
concentrations of imidazole-based polymerizable antibacterial monomer

| Antibacterial | Comparable | | | | |
|---|---|---|---|---|---|
| Orthodontic Resin | Example 1 | Example 1 | Example 2 | Example 3 | Example 4 |
| Resin Code | HLU14-114-SO | HLU14-196R1 | HLU14-182R | HLU14-196R2 | HLU14-183R |
| Antibacterial | 0 | 4 wt % | 8 wt % | 12 wt % | 16 wt % |
| Monomer in Resin | | | | | |
| Flexural Strength, | 97 (8) | 88 (5) | 91 (4) | 95 (3) | 81 (4) |
| MPa (s.d.) | | | | | |
| Flexural Modulus, | 2443 (87) | 2388 (183) | 2394 (139) | 2293 (82) | 2125 (125) |
| MPa (s.d.) | | | | | |

### Comparable Example 2

Conventional light curable orthodontic cement (HLU14-120P1, 75 wt % inorganic filler loading) without antibacterial monomer, was formulated, tested, and used as control orthodontic cement.

### Example 5

Light curable orthodontic cement (HLU14-197P1, 75 wt % inorganic filler loading) that contains 1 wt % polymerizable antibacterial monomer (ABR-C/XJ9-28) in cement was formulated and uniform paste was made on a ross mixer;

### Example 6

Light curable orthodontic cement (HLU14-184P1, 75 wt % inorganic filler loading) that contains 2 wt % polymerizable antibacterial monomer (ABR-C/XJ9-28) in cement was formulated and uniform paste was made on a ross mixer;

### Example 7

Light curable orthodontic cement (HLU14-197P2, 75 wt % inorganic filler loading) that contains 3 wt % polymerizable antibacterial monomer (ABR-C/XJ9-28) in cement was formulated and uniform paste was made on a ross mixer;

### Example 8

Light curable orthodontic cement (HLU14-184P2, 75 wt % inorganic filler loading) that contains 4 wt % polymerizable antibacterial monomer (ABR-C/XJ9-28) in cement was formulated and uniform paste was made on a ross mixer;

TABLE 3
Adhesive and physical properties of orthodontic Cements that contain various
concentrations of imidazole-based polymerizable antibacterial monomer

| Antibacterial | Comparable | | | | |
|---|---|---|---|---|---|
| Orthodontic Cement | Example 2 | Example 5 | Example 6 | Example 7 | Example 8 |
| Cement Code | HLU14-120P1 | HLU14-197P1 | HLU14-184P1 | HLU14-197P2 | HLU14-184P2 |
| Antibacterial | 0 | 1 wt % | 2 wt % | 3 wt % | 4 wt % |
| Monomer in Cement | | | | | |
| Resin Conc. | 25.0% | 25.0% | 25.0% | 25.0% | 25.0% |
| Ambient Light | 2:35″ | 2:00″ | 2:05″ | 2:00″ | 2:15″ |
| Sensitivity | | | | | |
| Compressive Strength, | 373 (25) | 361 (12) | 346 (10) | 352 (5) | 307 (15) |
| MPa (s.d.) | | | | | |
| Flexural Strength, | 150 (14) | 139 (6) | 114 (10) | 114 (10) | 88 (7) |
| MPa (s.d.) | | | | | |
| Flexural Modulus, | 11165 (531) | 10848 (383) | 10319 (549) | 10607 (629) | 10066 (445) |
| MPa (s.d.) | | | | | |
| NE-SBS to Etched | 32.8 (2.7) | 36.7 (4.5) | 28.8 (4.3) | 25.4 (3.5) | 31.0 (5.8) |
| Enamel, MPa (s.d.) | | | | | |
| SBS to Enamel Range, Mpa | 29.5~37.2 | 31.9~43.4 | 21.9~32.7 | 21.5~29.5 | 23.1~38.0 |
| SBS to Enamel C.V. | 8.1% | 12.1% | 14.8% | 14.0% | 18.7% |

As showed in Table 3, for the light curable orthodontic cements that contains 1-4 wt % polymerizable antibacterial monomer (ABR-C/XJ9-28) (Example 5 to Example 8), there are no drastic compromise to the ambient light sensitivity due to the adding of antibacterial monomer, and the values for Example 5 to Example 8 are comparable or better than the commercially available orthodontic cements products, as shown in FIG. 3.

As also showed in Table 3, for the light curable orthodontic cements that contains 1-3 wt % polymerizable antibacterial monomer (ABR-C/XJ9-28) (Example 5 to Example 7), there are no significant decrease to the compressive strength due to the incorporation of antibacterial monomer, and their compressive strength are all higher than the commercially available orthodontic cements products, as shown in FIG. 4. Slight decrease of compressive strength was observed when 4 wt % antibacterial monomer was incorporate (Example 8), but still comparable to commercially available orthodontic cements products.

As exhibited in Table 3, for the light curable orthodontic cements that contains 1-3 wt % polymerizable antibacterial monomer (ABR-C/XJ9-28) (Example 5 to Example 7), there are no drastic compromise to the flexural modulus due to the incorporation of antibacterial monomer, and values for Example 5 to Example 8 are comparable to the commercially available orthodontic cements products, except for the Light Bond from Reliance Orthodontics, as shown in FIG. 5.

Shear bond strength is an important property to evaluate the bonding performance of the orthodontic cement. As showed in Table 3, for the light curable orthodontic cements that contains 1-4 wt % polymerizable antibacterial monomer (ABR-C/XJ9-28) (Example 5 to Example 8), there are no significant compromise to the shear bond strength due to the incorporation of antibacterial monomer, and their shear bond strength are all comparable or higher than the commercially available orthodontic cements products, as shown in FIG. 6.

TABLE 4
ISO-22196 Antimicrobial Test using S. aureus 6538. The limit of detection
for this assay is 5 CFU/Carrier. Values below the limit of detection
are noted as <5.00E+00 CFU in the table and zero in the graph.

| | | | | | |
|---|---|---|---|---|---|
| | | | | Percent | Log₁₀ |
| | | | | Reduction | Reduction |
| | | | | Compared to | Compared to |
| Test | Contact | Carrier | | Control at | Control at |
| Microorganism | Time | Type | CFU/Carrier | Contact Time | Contact Time |

| | | | | |
|---|---|---|---|---|
| S. aureus | Time Zero | Control | 1.00E+06 | N/A |
| 6538 | 24 Hours | ATL Control | 8.50E+05 | |

| | | | |
|---|---|---|---|
| HLU14- | 1.00E+01 | 99.9988% | 4.93 |
| 184P1, Lot: | | | |
| 012815a | | | |
| HLU14- | | | |
| 184P2, Lot: | 1.50E+01 | 99.998% | 4.75 |
| 012315b | | | |
| HLU14- | | | |
| 197P1, Lot | 4.72E+03 | 99.44% | 2.26 |
| 021615a | | | |
| HLU14- | | | |
| 197P2, Lot: | <5.00E+00 | >99.9994% | >5.23 |
| 021615b | | | |

Antibacterial test was also conducted at an independent and GLP complied testing institution. As shown in Table 4 and FIG. 7, ortho adhesive paste formulations that incorporated imidazolium-based dimethacrylate antibacterial monomer (ABR-C), ISO-22196 antimicrobial testing results against microorganism ATCC 6538 showed significant levels of antibacterial effects, when compared with control. Such highly effective bactericidal effects for the imidazolium-based polymerizable resins were very promising due to a relatively low level loading.

As comparison, conventional QAS-based polymerizable resins are less effective and high dose loading (up to 30%) are required, which usually lead to decreasing in mechanical property and increasing cytotoxicity. Furthermore, with optimized formulation design not only highly effective antibacterial activity can be achieved, but also excellent mechanical and adhesion properties are yielded as showed in Table 3 and FIG. 3-6. The highly effectiveness in antibacterial for the light curable orthodontic cements that contains polymerizable antibacterial monomer offers great potential to reduce or prevent the occurrence of white spot lesions during the orthodontic treatment, especially for the “non-compliant” or “poor-compliant” patient groups.

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

We claim:

1. A method, comprising the steps of:
a. Etching a tooth surface,
b. Rinsing and drying the tooth surface,
c. Applying an antibacterial orthodontic cement onto a substrate surface of an orthodontic appliance,
d. Positioning the orthodontic appliance onto the tooth surface,
e. Pressing the orthodontic appliance onto the tooth surface,
f. Removing any excess cement, and
g. Light curing the antibacterial orthodontic cement,
Thereby securing the orthodontic appliance onto the tooth surface.

2. The method according to claim 1, where it comprises polymerizable resin, organic and inorganic filler particles, photoinitiators, stabilizers, and 0.1 wt %˜10 wt % polymerizable antibacterial/antimicrobial monomers in total cement compositions.

3. The method according to claim 2, where it contain at least one or multiple imidazolium groups and at least one or multiple radically polymerizable groups as shown in the following formula:
(M-RX₁)ₘ-A-(X₂R′-I-CB)ₙ
M: free radical polymerizable moiety such as acrylamide, methacrylamide, vinyl, acrylate, methacrylate;
X₁, X₂: are optional and may be equal or different linkages, such as, amide, ether, ester;
R,R′: equal or different moieties, such as, aromatic or alkyl;
A: moieties such as aromatic or alkyl;
C: counter ion moieties such as bromine, iodine, chlorine, inorganic acids or organic acids;
B: moieties containing linear or branched alkyl group having 4-16 carbon atoms;
I: imidazole moiety or substituted imidazole moiety like imidazole, methyl-imidazole, where m and n are integers of at least 1 or can same or different from 1 to 6.

4. The method according to claim 1, exhibited no significant compromise in adhesion and mechanical properties, while displaying highly effective antibacterial effects against the Staphylococcus aureus (S. aureus), which is commonly used model for gram positive bacteria and known to demonstrate resistance to antibiotics.

5. The method according to claim 1, further comprising 5 to about 70 percent by weight of free-radically polymerizable resins that doesn't contain antibacterial moiety.

6. The method according to claim 1, further comprising a photoinitiator system.

7. The method according to claim 1, wherein the filler particles are present from about 50 percent to about 90 percent by weight of the dental composite composition.

8. The method according to claim 1, wherein the filler particles comprises a mixture of micron-sized radiopaque filler particles and nano-sized filler particles.

9. The method according to claim 1, wherein the orthodontic appliance is a bracket.

10. The method according to claim 1, wherein the antibacterial orthodontic cement prevent white spot lesions on the tooth surface wherever the antibacterial orthodontic cement comes into contact with the tooth surface.

11. An antibacterial orthodontic cement comprising a polymerizable resin, organic and inorganic filler particles, photoinitiators, stabilizers, and a polymerizable antibacterial monomer, wherein the antibacterial monomer has a formulation of:
(M-RX₁)ₘ-A-(X₂R′-I-CB)ₙ
M: free radical polymerizable moiety such as acrylamide, methacrylamide, vinyl, acrylate, methacrylate;
X₁, X₂: are optional and may be equal or different linkages, such as, amide, ether, ester, direct;
R,R′: equal or different moieties, such as, aromatic or alkyl;
A: moieties such as aromatic or alkyl;
C: counter ion moieties such as bromine, iodine, chlorine, inorganic acids or organic acids;
B: moieties containing linear or branched alkyl group having 4-16 carbon atoms;
I: imidazole moiety or substituted imidazole moiety like imidazole, methyl-imidazole, where m and n are integers of at least 1 or can same or different from 1 to 6.
